(19) United States
(12) Patent Application Publication
(10) Pub. No.: US 2021/0307740 A1
(43) Pub. Date: Oct. 7, 2021

(54) WOUND HEALING AID DEVICE

(71) Applicant: Patrice Richards, Stone Mountain, GA (US)

(72) Inventor: Patrice Richards, Stone Mountain, GA (US)

(21) Appl. No.: 17/221,097

(22) Filed: Apr. 2, 2021

Related U.S. Application Data
(60) Provisional application No. 63/004,588, filed on Apr. 3, 2020.

(51) Int. Cl. A61B 17/02; A61F 13/02; A61F 13/00

(52) U.S. Cl. CPC A61B 17/0293; A61F 13/00063; A61F 13/023

(57) ABSTRACT

A wound healing aid device including a spacer, a ring and pair of flaps. The spacer is configured for implantation in an open wound. The spacer is positioned in the open wound in an area below a top layer of the wound. The ring is attached to the spacer and is configured to adhere to a top layer of skin surrounding the wound area. The pair of flaps are each positioned at an opposite side of the ring. The pair of flaps are configured to overlap one another in a closed positioned and are configured to separate from one another in an open position. The pair of flaps shield the open wound in the closed position and allow access to the wound in the open position.

### CROSS REFERENCE TO RELATED APPLICATIONS

This application claims the benefit of priority of U.S. Provisional Application No. 63/004,588 filed on Apr. 3, 2020, the disclosure of which is incorporated herein by reference in its entirety.

### FIELD OF THE INVENTION

Embodiments described herein generally relate to healing aid devices, and more particularly to wound healing aid devices.

### BACKGROUND OF THE INVENTION

Healing aids, such as, bandages and casts, are a major part of medical treatment. However, these healing aids may close or cover wounds too early prior to assessing infection status of tissue loss. This results in the spread of infection and/or improper healing. Moreover, for deep open wounds, the internal layers of skin of the wound may take longer to heal than the outermost layer of skin and healing aids that completely cover such wounds prevent proper assessment. Hence, an improved healing aid is desired that allows a wound to stay open for a predetermined period to ensure optimal healing.

### BRIEF DESCRIPTION OF THE DRAWINGS

The various advantages of the embodiments of the present disclosure will become apparent to one skilled in the art by reading the following specification and appended claims, and by referencing the following drawings, in which:

FIG. 1 shows an exemplary view of a wound healing aid device according to an embodiment of the present disclosure.

### SUMMARY OF THE INVENTION

Exemplary embodiments disclosed herein describe a wound healing aid device including a spacer, a ring and pair of flaps. The spacer is configured for implantation in an open wound. The spacer is positioned in the open wound in an area below a top layer of the wound. The ring is attached to the spacer and is configured to adhere to a top layer of skin surrounding the wound area. The pair of flaps are each positioned at an opposite side of the ring. The pair of flaps are configured to overlap one another in a closed positioned and are configured to separate from one another in an open position. The pair of flaps shield the open wound in the closed position and allow access to the wound in the open position.

In some exemplary embodiments, the spacer is positioned in the open wound at the dermis layer of skin.

In some exemplary embodiments, the spacer includes an orifice which is configured to surround the wound area.

In some exemplary embodiments, the spacer operates as a temporary space holder to keep the wound open in the area where the spacer resides.

In some exemplary embodiments, the ring includes an adhesive to adhere the ring to the top layer of skin surrounding the open wound.

In some exemplary embodiments, the ring includes an orifice which is positioned in alignment with the orifice of the spacer.

In some exemplary embodiments, the pair of flaps each include an antimicrobial gauze.

### DETAILED DESCRIPTION

The disclosure will be described more fully hereinafter with reference to the accompanying drawings, in which exemplary embodiments of the disclosure are shown. This disclosure may, however, be embodied in many different forms and should not be construed as limited to the exemplary embodiments set forth herein. It will be apparent to persons skilled in the relevant art that various changes in form and detail can be made to various embodiments without departing from the spirit and scope of the present disclosure. Thus, the breadth and scope of the present disclosure should not be limited by any of the above-described exemplary embodiments but should be defined only in accordance with the following claims and their equivalents.

The present disclosure relates to a wound healing aid device (“the device”). The device may be adapted for use with an open wound. In other aspects, the device may be adapted for use with a deep open wound (e.g., at the dermis layer/subcutaneous tissue). The device has a spacer which is configured to keep the epidermis layer of a wound open to prevent the epidermis layer of the wound from healing too quickly (i.e., healing before the lower layer(s) is/are healed). Moreover, the device includes a pair of flaps to keep the open wound covered at the epidermis layer while the tissue below the epidermis layer is exposed while healing. This ensures that the wound will heal properly with a minimal likelihood of infection and/or significant scarring. The flaps may be separated from one another to provide access to the open wound when needed, such as, for example, for assessing the wound and for applying dressing or treatment to the wound.

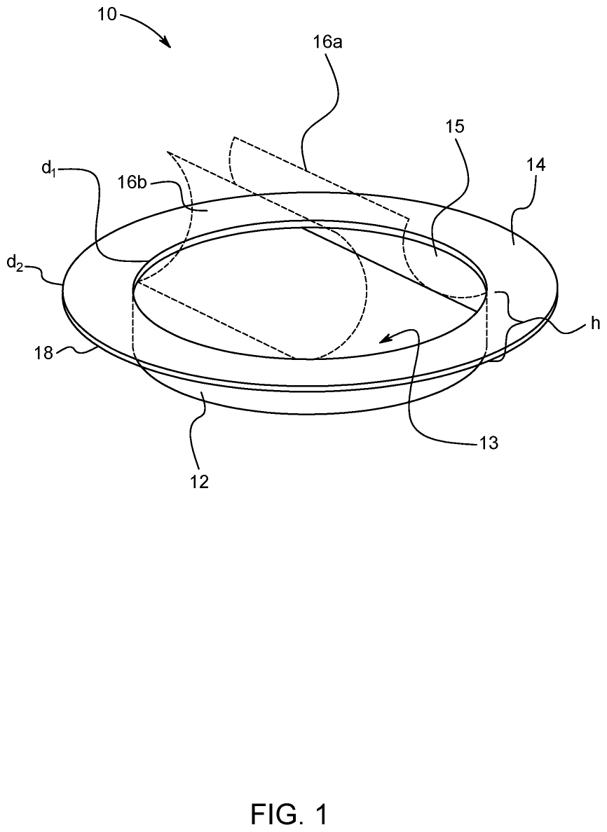

As illustrated in FIG. 1, the device 10 includes a spacer 12, a ring 14, a flap 16 (i.e., 16a and 16b) and an adhesive 18. The spacer 12 may have a circular shape and includes an orifice 13 which surrounds a wound area (i.e., the physical location on the human body where the wound exists). The spacer may be implanted in the open wound, and specifically the spacer may be positioned in one or more layers of skin above the muscle/connective tissue layer(s) of skin (e.g., the dermis layer or the subcutaneous layer) where the wound is located.

The spacer operates as a temporary space holder to keep the wound open in the area where the spacer resides, which allows the wound to stay open for a predetermined period to ensure optimal healing of the layers/tissue below the epidermis layer. Moreover, the spacer prevents the epidermis from healing too quickly. Thus, when the layers of skin below the spacer is healed (e.g., the dermis layer or the subcutaneous layer), the spacer is removed from the wound area. The spacer 12 may be made of any suitable material, such as, plastic, rubber, silicone. The spacer may be configured of any suitable size. However, the height (h) of spacer should be just tall enough to stop the epidermis from healing. In other words, the height of spacer should not fall short from the top layer of skin where the epidermis is.

The ring 14 may have a circular shape and may include an orifice 15 which surrounds the wound area at the outermost top layer of skin (e.g., epidermis). The ring's orifice is aligned with the orifice 13 of spacer 12. The ring has an inner diameter d₁ which is the same as the diameter of spacer 12. The ring 14 has an outer diameter d₂ which is larger than the diameter of spacer 12. The ring may be made of any suitable material. In a preferred embodiment, the ring is made of hard flexible silicone. The bottom surface of the ring includes an adhesive 18 for adhering the ring to a top layer of skin. The adhesive may include any suitable adhesive for adhering the ring to the top layer of skin.

The flap 16 includes a pair of overlapping protective flaps 16a and 16b which may be opened or closed. The flaps are each attached to a top surface of ring 14 along the edge of the orifice. Each flap is positioned at an opposite side of ring 14. In a closed position, the flaps overlap one another, thereby covering and shielding the open wound from contamination, infection, exposure to unwanted external substances. In an open position, the flaps are separated from one another, thereby allowing ease of assessment of the open wound area and treatment to the open wound area. In a default state, the flaps are in a closed position. The flaps may be made of any suitable material. In a preferred embodiment, the flaps are made of silicone. The flaps may each include a skin surface antimicrobial gauze (e.g., an antimicrobial dressing) for general cleaning, dressings, prepping, packing and debriding wounds. It can also be used as a temporary absorbent dressing over wounds.

The disclosed embodiments are not inclusive and many other modifications and variations will be apparent to someone of ordinary skill in the art with construction skills in the related arts. Together the descriptions and accompanying illustrations seek to provide an explanation of the basic principles of the embodiment and its application. It is therefore intended that the specification and embodiments be considered as exemplary only.

Those skilled in the art will appreciate from the foregoing description that the broad techniques of the embodiments of the present invention may be implemented in a variety of forms. Therefore, while the embodiments of this invention have been described in connection with particular examples thereof, the true scope of the embodiments of the invention should not be so limited since other modifications will become apparent to the skilled practitioner upon a study of the drawings, specification, and following claims.

1. A wound healing aid device comprising:
a spacer configured for implantation in an open wound, wherein the spacer is positioned in the open wound in an area below a top layer of the wound;
a ring attached to the spacer, the ring is configured to adhere to a top layer of skin surrounding the wound area; and
a pair of flaps each positioned at an opposite side of the ring, wherein the pair of flaps are configured to overlap one another in a closed positioned and are configured to separate from one another in an open position;
wherein the pair of flaps shield the open wound in the closed position and allow access to the wound in the open position.

2. The device of claim 1, wherein the spacer is positioned in the open wound at the dermis layer of skin.

3. The device of claim 1, wherein the spacer includes an orifice which is configured to surround the wound area.

4. The device of claim 1, wherein the spacer operates as a temporary space holder to keep the wound open in the area where the spacer resides.

5. The device of claim 1, wherein the ring includes an adhesive to adhere the ring to the top layer of skin surrounding the open wound.

6. The device of claim 2, wherein the ring includes an orifice which is positioned in alignment with the orifice of the spacer.

7. The device of claim 1, wherein the pair of flaps each include an antimicrobial gauze.
